(19) United States
(12) Patent Application Publication
(10) Pub. No.: US 2004/0126330 A1
(43) Pub. Date: Jul. 1, 2004

(54) Nicotine-containing film preparation

(76) Inventors: Tsutomu Awamura, Imizu-gun (JP); Yoshihiro Sawai, Imizu-gun (JP)

Correspondence Address: Edwards & Angell, PO Box 9169, Boston, MA, 02209, US

(21) Appl. No.: 10/475,790

(22) PCT Filed: Sep. 20, 2002
(86) PCT No.: PCT/JP02/09682

(30) Foreign Application Priority Data
Sep. 25, 2001 (JP) 2001-292616

(51) Int. Cl. A61K 9/68; A61K 31/4439

(52) U.S. Cl. 424/48; 514/343

(57) ABSTRACT

A film preparation of a three-layer structure which comprises: a nicotine-containing layer comprising nicotine and an edible water-soluble polymer and having a 180-degree peel strength in application to a Bakelite plate of 1 g to 30 g/20 mm and a solubility in water of 3 minutes to 10 minutes,

and two coating layers laminated respectively on both sides of the nicotine-containing layer, the coating layers comprising a water-soluble and non-water-absorbing polysaccharide and/or an edible polymer and a softener and the coating layers each having a 180-degree peel strength in application to a Bakelite plate of 100 g to 500 g/20 mm and a solubility in water of 0.5 minutes to 3 minutes.

### TECHNICAL FIELD

The present invention relates to a nicotine-containing film preparation for application to the oral mucous membrane, which is used as an aid for smoking cessation or moderation in smoking, and more particularly to a film preparation having a three-layered structure comprising a nicotine-containing drug layer and coating layers provided on both faces thereof, the coating layers being able to adhere to the oral mucous membrane.

### BACKGROUND ART

As for preparations relating to nicotine administration through the oral mucous membrane, certain preparations have been known. For example, there have been known tablets for intraoral or sublingual administration (for example, refer to patent documents 1 and 2), lozenges (for example, refer to patent documents 3 and 4), hydrocarbon powder matrixes (for example, refer to patent document 5), troches (for example, refer to patent document 6: specifically tablet type troches), capsules (for example, refer to patent document 7), saliva-soluble preparations including patches in claims, but no specific patch examples are seen at all (for example, refer to patent documents 8 and 9) and patches for the oral mucous membrane (for example, refer to patent documents 10, 11, 12 and 13).

### PRIOR ART

[Patent Document 1]

International Patent Unexamined Publication No.6-502622 (pages 1 and 2)

[Patent Document 2]

International Patent Unexamined PublicationNo. 5-504949 (pages 1 and 2)

[Patent Document 3]

International Patent Unexamined PublicationNo. 9-505553 (pages 1 and 2)

[Patent Document 4]

Japanese Patent Unexamined Publication No. 9-286729 (pages 1 and 2)

[Patent Document 5]

Japanese Patent Unexamined Publication No. 9-286729 (pages 1 and 2)

[Patent Document 6]

International Patent Unexamined Publication No. 2000-504028 (pages 1 and 2)

[Patent Document 7]

Japanese Patent No. 2602043 (pages 1 and 2)

[Patent Document 8]

Japanese Patent No. 2958663 (pages 1 and 2)

[Patent Document 9]

Japanese Patent Unexamined Publication No. 11-315023 (pages 1 and 2)

[Patent Document 10]

Japanese Patent Unexamined Publication No. 3-209626 (pages 1 and 2)

[Patent Document 11]

Japanese Patent Unexamined Publication No. 3-209627 (pages 1 and 2)

[Patent Document 12]

Japanese Patent Unexamined Publication No. 62-178513 (pages 1 and 2)

[Patent Document 13]

Japanese Patent Unexamined Publication No. 63-227522 (pages 1 and 2)

Although the conventional nicotine administration preparations through the oral mucous membrane have each been cogitated, many have a feeling that something is wrong. Further, the preparations are easily crunched or swallowed, or complicated in their structure. Accordingly, there is the problem of complicated manufacturing methods. Furthermore, the preparations have insufficient sustained release, poor reproducibility or the difficulty of controlling sustained release.

When nicotine is allowed to be absorbed through the oral mucous membrane, the dissolution time of a film preparation is not particularly specified. However, when it dissolves too early, the active ingredient is difficult to stay in the oral cavity, resulting in insufficient absorption. Admitting that the feeling that something is wrong is decreased, adhesion to the upper jaw for a period longer than necessary raises the problem that a disturbance is unfavorably caused by the taste of nicotine itself etc. in the case of drinking and eating during that.

The present invention provides a nicotine film preparation easily adhesive to the mucous membrane, having a suitable sustained release of nicotine and good in reproducibility thereof, rich in flexibility and having elasticity, and having no feeling that something is wrong in application.

### DISCLOSURE OF THE INVENTION

In order to solve the above-mentioned problems, that is to say, in order to provide a nicotine-containing film preparation having suitable sustained release and no feeling that something is wrong in application, the present inventors have made intensive studies. As a result, the inventors have discovered that a film preparation having suitable sustained release, having no feeling that something is wrong in application, and easily adhesive to the mucous membrane is obtained by combining coating layers having a specified peel strength and solubility and a nicotine-containing layer having a specified peel strength and solubility different from those of the coating layers so as to give a definite thickness, thus completing the present invention.

That is to say, the present invention is directed to (1) a film preparation of a three-layer structure which comprises: a nicotine-containing layer comprising nicotine and an edible water-soluble polymer and having a 180-degree peel strength in application to a Bakelite™ plate of 1 g to 30 g/20 mm and a solubility in water of 3 minutes to 10 minutes,

and two coating layers laminated respectively on both sides of the nicotine-containing layer, the coating layers comprising a water-soluble and non-water-absorbing polysaccharide and/or an edible polymer and a softener and the coating layers each having a 180-degree peel strength in application to a Bakelite plate of 100 g to 500 g/20 mm and a solubility in water of 0.5 minutes to 3 minutes; (2) the film preparation described in (1), in which the pH of a 0.1% aqueous solution of the preparation is from 3 to 9; (3) the film preparation described in (1), in which the nicotine content in one preparation unit is from 0.1 mg to 5 mg; (4) the film preparation described in (1), in which the time required for dissolution of one preparation unit is from 10 minutes to 30 minutes; (5) the film preparation described in (1), which has a film strength of 2 kg to 5 kg/20 mm; (6) the film preparation described in (1), in which the nicotine-containing layer has a thickness of 250 μm to 500 μm, the coating layer has a thickness of 5 μm to 30 μm, and a total of the three layers have a thickness of 260 μm to 560 μm; (7) the film preparation described in (1), in which the coating layer contains a sucrose fatty acid ester; (8) the film preparation described in (1), in which the nicotine-containing layer contains polyethylene glycol; and (9) the film preparation described in (1), in which the water-soluble and non-water-absorbing polysaccharide is at least one of water-soluble starch, pullulan, a pullulan ether (methyl, ethyl or propyl) and an pullulan ester (acetate or butyrate), the softener is at least one of glycerol, ethylene glycol, propylene glycol, D-sorbitol, maltitol, mannitol and xylitol, the edible water-soluble polymer is at least one of hydroxypropylcellulose (HPC), hydroxypropylmethyl cellulose (HPMC), hydroxypropylethylcellulose (HPEC), methyl cellulose (MC), water-soluble ethyl cellulose (EC), water-soluble hydroxyethyl cellulose (HEC), carboxymethyl cellulose-sodium (CMC-Na), polyvinyl alcohol (PVA), polyvinyl pyrrolidone (PVP), a carboxyvinyl polymer, sodium alginate and sodium acrylate, and the edible polymer is at least one of hydroxypropylcellulose (HPC), hydroxypropylmethyl cellulose (HPMC), hydroxypropylethyl cellulose (HPEC), methyl cellulose (MC), ethyl cellulose (EC), hydroxyethyl cellulose (HEC), carboxymethyl cellulose-sodium (CMC-Na), polyvinyl alcohol (PVA), polyvinyl pyrrolidone (PVP), a carboxyvinyl polymer, sodium alginate, sodium acrylate, shellac, cellulose acetate phthalate (CAP), polyvinyl acetate, Eudragit™, hydroxypropylmethyl cellulose acetate succinate (HPMCAS) and hydroxypropylmethyl cellulose phthalate (HPMCP).

Previously, Japanese Patent Unexamined Publication No. 3-209626 (patent document 10), Japanese Patent Unexamined Publication No. 3-209627 (patent document 11), Japanese Patent Unexamined Publication No. 62-178513 (patent document 12) and Japanese Patent Unexamined Publication No. 63-227522 (patent document 13) have been mentioned as ones relating to the nicotine-containing patches for the oral mucous membrane.

In all of the preparations of these inventions, water-soluble or water-swellable polymers are used, and there may be particularly no problem in respect to adhesion to the mucous membrane (in the experiments, tests are carried out by sticking the preparations on beaker walls, and this is different from actual use). However, the time for which the preparations have been applied is considered to be too long, and the initial release of drugs is considered to be insufficient. The film preparation of the present invention completely dissolves in 10 to 30 minutes, gives neither a feeling that something is wrong nor an uncomfortable feeling, and allows nicotine to be sufficiently absorbed.

### BEST MODE FOR CARRYING OUT THE INVENTION

The present invention will be described in more detail below.

In the present invention, a coating layer comprises a water-soluble and non-water-absorbing polysaccharide and/or an edible polymer and a softener. The coating layer is mainly composed of pullulan or the like, the water-soluble and non-water-absorbing polysaccharide, so that it plays a role for protecting a surface of a product. The film preparation of the present invention has thin layers mainly composed of pullulan on both upper and lower faces. Accordingly, when pressed onto the upper jaw, the coating layer dissolves immediately (0.5 to 3 minutes) by moisture of the upper jaw portion, and a nicotine-containing layer comes into contact with the upper jaw and adheres thereto. It does not therefore happen to separate immediately. Then, this nicotine-containing layer dissolves in 10 to 30 minutes to release nicotine, which is absorbed through the mucous membrane.

Although the water-soluble and non-water-absorbing polysaccharides used in the present invention include water-soluble starch, pullulan, a pullulan ether (methyl, ethyl or propyl) and a pullulan ester (acetate or butyrate), pullulan is suitably used. Although the softeners include glycerol, ethylene glycol, propylene glycol, D-sorbitol, maltitol, mannitol, xylitol, etc., D-sorbitol is suitably used. There is no particular limitation on the edible water-soluble polymer, and examples thereof include hydroxypropylcellulose (HPC), hydroxypropylmethyl cellulose (HPMC), hydroxypropyl-ethylcellulose (HPEC), methyl cellulose (MC), water-soluble ethyl cellulose (EC), water-soluble hydroxyethyl cellulose (HEC), carboxymethyl cellulosesodium (CMC-Na), polyvinyl alcohol (PVA), polyvinyl pyrrolidone (PVP), a carboxyvinyl polymer, sodium alginate, etc. HPC and PVP (particularly, polyvinyl pyrrolidone K-90) are suitably used. The edible polymers include water-insoluble polymers such as water-insoluble ethyl cellulose, water-insoluble hydroxyethyl cellulose, shellac, cellulose acetate phthalate (CAP) and polyvinyl acetate, and water-swellable polymers such as Eudragit, hydroxypropylmethyl cellulose acetate succinate (HPMCAS) and hydroxypropylmethyl cellulose phthalate (HPMCP), as well as the above-mentioned edible water-soluble polymers. Although polyethylene glycol includes Macrogol 300 and Macrogol 400, Macrogol 400 is suitable. In the present invention, a sweetener and a flavor can be used, in addition to the above-mentioned ingredients. As the sweeteners, there can be used saccharine, saccharin sodium, aspartame, stevioside, sugar, fructose, glucose, etc. As the flavors, various kinds of flavors can be used.

A nicotine-containing layer of the present invention comprises nicotine and an edible water-soluble polymer.

Nicotine used in the invention is desirably one having a grade of 99% or more. Low-grade nicotine is unsuitable because of its strong stimulation. The nicotine content in one preparation unit is from 0.1 to 5 mg. There is no particular limitation on the edible water-soluble polymer, a main adhesive ingredient, and examples thereof include hydroxypropylcellulose (HPC), hydroxypropylmethyl cellulose (HPMC), hydroxypropylethylcellulose (HPEC), methyl cellulose (MC), water-soluble ethyl cellulose (EC), water-soluble hydroxyethyl cellulose (HEC), carboxymethyl cellulose-sodium (CMC-Na), polyvinyl alcohol (PVA), polyvinyl pyrrolidone (PVP), a carboxyvinyl polymer, sodium alginate, etc. HPC and PVP (particularly, polyvinyl pyrrolidone K-90) are suitably used. The compounding ratio of the edible water-soluble polymer is from 40 to 90% based on the whole preparation. That of the sweetener is preferably from 0.2 to 0.8% as well, the compounding ratio of the water-soluble and non-water-absorbing polysaccharide is preferably from 3 to 6% as well, that of the softener is preferably from 0.5 to 2% as well, and the compounding ratio of the edible polymer is preferably from 3 to 6% as well. Further, that of polyethylene glycol is preferably from 1 to 10% as well.

In this film preparation, the thickness of the coating layer is suitably from 5 to 30 μm, and the thickness of the nicotine-containing layer is suitably from 250 to 500 μm. The thickness as a whole is suitably from 260 to 560 μm. When the coating layer becomes too thick, the flexibility of the preparation is lost. Although it easily dissolves, some time is required because of its thickness, resulting in the difficulty of smoothly adhering to the inside of the oral cavity (upper jaw). Conversely, when it is too thin, unevenness is liable to occur in thickness in coating. In the extreme case, this introduces the possibility of some portions being not coated. In such a case, the protection of the nicotine-containing layer becomes insufficient. When the nicotine-containing layer becomes too thick, the time required for dissolution is unfavorably prolonged. Conversely, when it is thin, the duration is shortened, resulting in the possibility of the beneficial effect being not sufficiently exhibited.

As for the preparation like the present invention, there is no particular requirement on the time required for dissolution. However, the use of materials having properties as described above and the thickness as described above allow the preparation of the present invention to dissolve in 10 to 30 minutes. Faster than 10 minutes results in insufficient absorption of nicotine through the mucous membrane, whereas slower than 30 minutes results in the occurrence of various disadvantages.

The peeling strength in application to a Bakelite™ plate of the nicotine-containing layer and the coating layer of the film preparation of the present invention are from 3 to 10 minutes and from 0.5 to 3 minutes, respectively. The peeling strength in application to a Bakelite plate was measured in the following manner. A nicotine-containing layer patch or a coating layer patch is spread on a PET film having a thickness of 10 μm to prepare a film having a thickness of 100 μm. The film is cut to a size of 2×5 cm to prepare a test piece. A patch face of the test piece is uniformly impregnated with 200 μml of purified water, and then stuck on the Bakelite plate. A roller having a weight of 850 g is applied onto the Bakelite plate from above to adhere the patch face to the Bakelite plate. Then, a periphery of the test piece is pinched with a clip connected to a weight-measuring unit of a rheometer, and a test table is allowed to go down at a speed of 20 cm/min. Thus, the peeling strength of each patch to the Bakelite plate is determined (as the rheometer, Type RT-302OD-CW manufactured by Leotec Co. was used).

The peeling strength of the nicotine-containing layer in application to the Bakelite plate is suitably from 1 to 30 g/20 mm. There are the problems that less than 1 g/20 mm results in insufficient compatibility with the coating layer, whereas more than 30 g/20 mm results in the difficulty of production, the difficulty of separating from the base film when spread, and the difficulty of adhering to the inside of the oral cavity (upper jaw) even when tentatively used as a product.

Further, the peeling strength of the coating layer to the Bakelite plate is suitably from 100 to 500 g/20 mm. Less than 100 g/20 mm results in the difficulty of adhering to the inside of the oral cavity (upper jaw), whereas more than 500 g/20 mm results in the difficulty of production.

The solubility in water was measured in the following manner.

A nicotine-containing layer and a coating layer are each prepared so as to form a 100-μm film in the same manner as described above. These are cut to a size of 1.5×1.5 cm to prepare test pieces. Each test piece is taken, and tested at 50 revolutions with a sinker by The Pharmacopoea of Japan, the general test method, the elution test method, the second method, using 900 mL of purified water as a test solution. Complete dissolution is visually confirmed, and the time required for dissolution is taken as the solubility. The solubility in water of the nicotine-containing layer is suitably from 3 to 10 minutes. Faster than 3 minutes results in no expectation of the nicotine effect, whereas slower than 10 minutes results in the possibility of experiencing a feeling that something is wrong in the oral cavity. Further, the solubility in water of the coating layer is suitably from 0.5 to 3 minutes. Faster than 0.5 minute or slower than 3 minutes results in the difficulty of adhering to the inside of the oral cavity (upper jaw).

The pH of the film preparation of the present invention (a 0.1% aqueous solution) is suitably from 3 to 9. One sheet of this preparation is dissolved in 10 ml of purified water, and subjected to a measurement. When the pH is lower than 3 or higher than 9, stimulation to the mucous membrane is liable to occur.

The time required for dissolution of the film preparation of the present invention is suitably from 10 to 30 minutes as described above.

For the time required for dissolution, one sheet of this preparation was stuck on the upper jaw of each of healthy volunteers (7 volunteers), and the time required for complete dissolution was measured (sensory evaluation).

The strength of the film preparation of the present invention is suitably from 2 to 5 kg/20 mm.

This film preparation is cut to a size of 2×5 cm (measuring length: 3 cm) to prepare a test piece. A periphery (2 cm in width) of the test piece was connected to a weight measuring unit of a rheometer, and a test table is allowed to go down at a speed of 20 cm/min, thereby breaking this film preparation. The breaking strength required at that time is measured. When the strength is weaker than 2 kg/20 mm, the expected time required for dissolution is not obtained. Stronger than 5 kg/20 mm results in the possibility of the film cracking.

### EXAMPLES

Examples and reference examples are shown below, but the invention should not be construed as being limited to these examples.

### Example 1

To an appropriate amount of purified water, 79.5 g of pullulan, 20.0 g of D-sorbitol and 0.5 g of a sucrose fatty acid ester are added, and dissolved by stirring to prepare a coating layer solution. Separately, 2.0 g of nicotine, 4.0 g of titanium oxide, 8.0 g of Macrogol 400, 1.0 g of 1-menthol, 0.5 g of saccharin sodium, 0.1 g of a flavor and 84.4 g of hydroxypropylcellulose (L) are added to an appropriate amount of ethanol, and dissolved by stirring to prepare a nicotine-containing layer solution. Then, the coating layer solution is spread on a polyester release film, and dried to form a film having a thickness of about 10 μm. The nicotine-containing layer solution is spread thereon, and dried to prepare a film having a thickness of about 330 μm. Finally, the coating layer solution is spread thereon, and dried to form a film having a thickness of about 10 μm. The layers are thus laminated to prepare a film-shaped product having a thickness of about 350 μm as a whole. This was stamped out to a circular form having a diameter of 12 mm, thereby obtaining a nicotine-containing film preparation.

Hereinafter, Examples 2 and 3 and Comparative Examples and 2 were carried out similarly to Example 1. Results hereof are shown in Table 1.

TABLE 1

| | | | | | | | Comparative |
|---|---|---|---|---|---|---|---|
| Layer | Name of Raw Material | | Example 1 | Example 2 | Example 3 | Example 1 | Example 2 |
| Coating | Pullulan PI-20 | | 79.5 | | | | 22.0 |
| Layer | D-sorbitol | | 20.0 | | | | 75.0 |
| | Sucrose Fatty Acid Ester | | 0.5 | | | | 3.0 |
| Nicotine- | Nicotine | | 2.0 | 2.0 | 2.0 | 2.0 | 2.0 |
| Containing | Titanium Oxide | | 4.0 | | 4.0 | | |
| Layer | PEG 400 | | 8.0 | 5.0 | 5.0 | 10.0 | 10.0 |
| | HPC-L | | 84.4 | 51.0 | 82.4 | 38.0 | 38.0 |
| | PVP K90 | | | 40.0 | | | |
| | PVP K30 | | | | | 50.0 | 50.0 |
| | Ethyl Cellulose | | | | | | |
| | 1-Menthol | | 1.0 | | 1.0 | | |
| | Saccharin Sodium | | 0.5 | | 0.5 | | |
| | Citric Acid | | | 2.0 | 5.0 | | |
| | Flavor | | | 0.1 | | 0.1 | |
| Claim 1 | Nicotine- | Peel strength | 5.6 | 5.0 | 5.3 | 35.0 | 35.0 |
| | Containing | (g/20 mm) | | | | | |
| | Layer | Solubility (mm) | 5.0 | 3.8 | 5.0 | 2.8 | 2.8 |

| | | | |
|---|---|---|---|
| Coating | Peel strength | 296.7 | 85.0 |
| Layer | (g/20 mm) | | |
| | Solubility (min) | 1.5 | 0.4 |

| Claim 2 | PH | 8.85 | 6.80 | 3.56 | 9.20 | 9.20 |
|---|---|---|---|---|---|---|
| Claim 3 | Nicotine Content (mg) | 1.0 | | | | |
| Claim 4 | Time Required for Dissolution | 21.0 | 18.0 | 20.9 | 9.0 | 8.0 |
| | (min) | | | | | |
| Claim 5 | Strength (kg/20 mm) | 2.8 | 2.6 | 3.0 | 1.8 | 1.7 |
| Claim 6 | Nicotine-Containing Layer (μm) | 350 | 370 | 350 | 390 | 380 |

| | |
|---|---|
| Coating Layer (μm) | 10 |

### Comparative Example 1

The solubility of the nicotine-containing layer is high (2.8 minutes), so that the time required for dissolution becomes faster than the proper time (9.0 minutes), which poses a problem with regard to sustained release. The pH is high, so that stimulation is given to the mucous membrane. The peeling strength is so high that the production thereof is difficult. For example, when spread on a PET film, it becomes difficult to-be separated from the PET film.

### Comparative Example 2

The peel strength of the coating layer is low, and the adhesive force is low. Accordingly, adhesion to the upper jaw becomes difficult. The others are similar to Comparative Example 1.

### EFFECT OF THE INVENTION

The nicotine film preparation of the present invention is low in solubility, has a sustained release, and is moderate in the peeling strength of the nicotine-containing layer and the peeling strength of the coating layer, so that it easily adheres to the mucous membrane. Further, the nicotine film preparation of the present invention has a pH of 3 to 9, has no stimulation to the mucous membrane, is rich in flexibility and has elasticity, and has no feeling that something is wrong in application.

Industrial Applicability

The nicotine film preparation of the present invention easily adheres to the mucous membrane, has a suitable sustained release of nicotine and is good in reproducibility thereof, is rich in flexibility and has elasticity, and has no feeling that something is wrong in application. Thus, the preparation is useful as a nicotine-containing film preparation for application to the oral mucous membrane, which is used as an aid for smoking cessation or moderation in smoking.

1. A film preparation of a three-layer structure which comprises: a nicotine-containing layer comprising nicotine and an edible water-soluble polymer and having a 180-degree peel strength in application to a Bakelite plate of 1 g to 30 g/20 mm and a solubility in water of 3 minutes to 10 minutes, and two coating layers laminated respectively on both sides of the nicotine-containing layer, the coating layers comprising a water-soluble and non-water-absorbing polysaccharide and/or an edible polymer and a softener and the coating layers each having a 180-degree peel strength in application to a Bakelite plate of 100 g to 500 g/20 mm and a solubility in water of 0.5 minutes to 3 minutes.

2. The film preparation according to claim 1,in which the pH of a 0.1% aqueous solution of the preparation is from 3 to 9.

3. The film preparation according to claim 1, in which the nicotine content in one preparation unit is from 0.1 mg to 5 mg.

4. The film preparation according to claim 1, in which the time required for dissolution of one preparation unit is from 10 minutes to 30 minutes.

5. The film preparation according to claim 1, which has a film strength of 2 kg to 5 kg/20 mm.

6. The film preparation according to claim 1, in which the nicotine-containing layer has a thickness of 250 μm to 500 μm, the coating layer has a thickness of 5 μm to 30 μm, and a total of the three layers have a thickness of 260 μm to 560 μm.

7. The film preparation according to claim 1, in which the coating layer contains a sucrose fatty acid ester.

8. The film preparation according to claim 1, in which the nicotine-containing layer contains polyethylene glycol.

9. The film preparation according to claim 1, in which the water-soluble and non-water-absorbing polysaccharide is at least one of water-soluble starch, pullulan, a pullulan ether (methyl, ethyl or propyl) and an pullulan ester (acetate or butyrate); the softener is at least one of glycerol, ethylene glycol, propylene glycol, D-sorbitol, maltitol, mannitol and xylitol; the edible water-soluble polymer is at least one of hydroxypropylcellulose (HPC), hydroxypropylmethyl cellulose (HPMC), hydroxypropylethylcellulose (HPEC), methyl cellulose (MC), water-soluble ethyl cellulose (EC), water-soluble hydroxyethyl cellulose (HEC), carboxymethyl cellulose-sodium (CMC-Na), polyvinyl alcohol (PVA), polyvinyl pyrrolidone (PVP), a carboxyvinyl polymer, sodium alginate and sodium acrylate; and the edible polymer is at least one of hydroxypropylcellulose (HPC), hydroxypropylmethyl cellulose (HPMC), hydroxypropylethyl cellulose (HPEC), methyl cellulose (MC), ethyl cellulose (EC), hydroxyethyl cellulose (HEC), carboxymethyl cellulose-sodium (CMC-Na), polyvinyl alcohol (PVA), polyvinyl pyrrolidone (PVP), a carboxyvinyl polymer, sodium alginate, sodium acrylate, shellac, cellulose acetate phthalate (CAP), polyvinyl acetate, Eudragit, hydroxypropylmethyl cellulose acetate succinate (HPMCAS) and hydroxypropylmethyl cellulose phthalate (HPMCP).
